(19) United States
(12) Patent Application Publication
(10) Pub. No.: US 2025/0177596 A1
(43) Pub. Date: Jun. 5, 2025

(54) POLYESTER SOLID ARTICLE COMPRISING HYDROPHOBIC MATERIAL

(71) Applicant: The Procter & Gamble Company, Cincinnati, OH (US)

(72) Inventors: Susana FERNANDEZ PRIETO, Benicarlo (ES); Johan SMETS, Lubbeek (BE); Isabelle GUIMET, Uccle (BE); Peter DE NIES, Putte (BE); Domenico PIRONE, Bruxelles (BE); Jose Blas MARTINEZ HERNANDEZ, Wezembeek - Oppem (BE); Gaurav SAINI, Singapore (SG); Luke Andrew ZANNONI, West Chester, OH (US); Esperanza CORTES TRIVINO, Huelva (ES); Inmaculada MARTINEZ GARCIA, Huelva (ES)

(21) Appl. No.: 18/956,691

(22) Filed: Nov. 22, 2024

Related U.S. Application Data
(60) Provisional application No. 63/604,927, filed on Dec. 1, 2023.

(51) Int. Cl. A61L 9/04; A61L 101/46

(52) U.S. Cl. CPC A61L 9/042; A61L 9/048; A61L 2101/46; A61L 2209/135

(57) ABSTRACT

Provided herein is a solid article for sustained release of one or more hydrophobic materials, comprising at least 5 wt. % of one or more hydrophobic materials embedded in a gel matrix,
wherein said gel matrix is formed from a chemically cross-linked polyester material.

### FIELD OF THE INVENTION

The present invention relates to a solid article for the sustained release of one or more hydrophobic materials, particularly of volatile hydrophobic materials such as perfumes, insect repellents, essential oils, functional perfume components (FPCs), aesthetic agents, bioactive agents, malodor counteractants, and mixtures thereof.

### BACKGROUND OF THE INVENTION

It is generally known to use a device to evaporate a volatile material into a space, particularly a domestic space, in order to deliver a variety of benefits, such as air freshening or perfuming of the air. Non-energized systems, for example, systems that are not powered by electrical energy, are a popular way for the delivery of volatile materials into the atmosphere.

These systems can be classified into those that require human actuation, such as aerosols, and those which do not require human actuation, such as wick-based systems and gels. The first type delivers the volatile materials on demand and the second type in a more continuous manner.

Variations on the second type of systems include polymer gel-based systems in which a volatile material is held within the matrix of a polymer gel. The volatile material may slowly be released from the polymer gel, providing sustained release over a desirable time period. Such products are disclosed in U.S. Pat. No. 11,173,224B2. However, these products are based on polyurethane polymer gels, which results in a number of disadvantages. First, the production of such polyurethane polymer gels requires the use of isocyanate starting materials, which may cause health problems in exposed humans. This requires significant safety precautions during manufacture, increasing costs. Second, the polyurethane polymer gels themselves have low biodisintegration/compostability, resulting in sustainability concerns.

There is a need for improved products that overcome some or all of the problems associated with the prior art.

### SUMMARY OF THE INVENTION

It has surprisingly been found that a polymer gel based on polyester chemistry may provide excellent release of one or more hydrophobic materials, especially volatile hydrophobic materials.

As compared to polyurethane-based gels, polyester-based gels may be prepared from advantageously less harmful starting materials, and the resulting polyester has improved biodisintegration.

Thus, the present invention provides the following.

1. A solid article for sustained release of one or more hydrophobic materials, comprising at least 5 wt. % of one or more hydrophobic materials embedded in a gel matrix,

wherein said gel matrix is formed from a chemically cross-linked polyester material.

2. The solid article of clause 1, wherein said chemically cross-linked polymeric material is a polyester obtainable by cross-linking an epoxidized vegetable oil with a polyfunctional carboxylic acid.

3. The solid article of clause 2, wherein said epoxidized vegetable oil comprises molecules having more than one (e.g. from 2 to 8, such as from 2 to 6) epoxy groups, and where the epoxidized vegetable oil is characterized by an oxirane oxygen content of at least 5% (e.g. at least 6%);

wherein optionally said epoxidized vegetable oil is selected from the group consisting of epoxidized linseed oil, epoxidized soybean oil, epoxidized castor oil, epoxidized rapeseed oil, epoxidized vernonia oil, vernonia oil, epoxidized corn oil, epoxidized cottonseed oil, epoxidized canola oil, epoxidized grape seed oil, epoxidized poppy seed oil, epoxidized tung oil, epoxidized sunflower oil, epoxidized safflower oil, epoxidized walnut oil, and any combinations thereof;

and wherein more optionally said epoxidized vegetable oil is epoxidized soybean oil.

4. The solid article of clause 2 or 3, wherein said polyfunctional carboxylic acid is soluble in at least one non-alcoholic (e.g. non-nucleophilic) solvent;

optionally wherein said polyfunctional carboxylic acid is selected from the group consisting of citric acid, maleic acid, malonic acid, itaconic acid, fumaric acid, succinic acid, methyl succinic acid, malic acid, phthalic acid, tartaric acid, citraconic acid, furan dicarboxylic acid, and any combinations thereof;

more optionally wherein said polyfunctional carboxylic acid is selected from the group consisting of citric acid, maleic acid, itaconic acid, fumaric acid, succinic acid, methyl succinic acid, malic acid, tartaric acid, citraconic acid, furan dicarboxylic acid, and any combinations thereof; and

more optionally still wherein said polyfunctional carboxylic acid is citric acid.

5. The solid article according to any one of clauses 2-4, wherein said polyester is obtainable from a crosslinking reaction carried out in the presence of a non-alcoholic solvent;

wherein optionally said non-alcoholic solvent is selected from the group consisting of water, acetone, toluene, and any combinations thereof; and

wherein optionally said non-alcoholic solvent is water.

6. The solid article according to any one of the preceding clauses, wherein the solid article is self-supporting.

7. The solid article according to any one of the preceding clauses, wherein the solid article comprises from 5 to 85 wt. % of the one or more hydrophobic materials, based on the total mass of the solid article,

optionally from 10 to 75 wt. %,

more optionally from 15 to 60 wt. %,

further optionally from 20 to 55 wt. %,

more optionally still from 20 to 50 wt. %.

8. The solid article according to any one of the preceding clauses, wherein the one or more hydrophobic materials have a logP of from 0.01 to 6.5 on a weighted average basis,

optionally from 0.5 to 5.5,

more optionally from 1.5 to 5.5,

further optionally from 2 to 5.

9. The solid article according to any one of the preceding clauses, wherein the one or more hydrophobic materials comprise one or more volatile hydrophobic materials,
optionally wherein the one or more hydrophobic materials comprise from 20 to 100 wt. % of one or more volatile hydrophobic materials,
optionally wherein the one or more hydrophobic materials comprise from 40 to 100 wt. % of one or more volatile hydrophobic materials,

optionally wherein the one or more hydrophobic materials comprise from 60 to 100 wt. % of one or more volatile hydrophobic materials,

optionally wherein the one or more hydrophobic materials comprise from 80 to 100 wt. % of one or more volatile hydrophobic materials.

10. The solid article according to clause 9, wherein the one or more volatile hydrophobic materials each have a boiling point of less than or equal to 450° C. at atmospheric pressure,
more optionally wherein the volatile hydrophobic material has boiling point of from 60° C. to 400° C. at atmospheric pressure,
further optionally wherein the volatile hydrophobic material has boiling point of from 75° C. to 380° C. at atmospheric pressure.

11. The solid article according to clause 9 or 10, wherein the one or more volatile hydrophobic materials each have a vapor pressure of at least 10⁻⁶ Torr at 25° C. and atmospheric pressure,
optionally wherein the volatile hydrophobic material has a vapor pressure of at least 10⁻⁵ Torr at 25° C.,
more optionally wherein the volatile hydrophobic material has a vapor pressure of at least 10⁻⁴ Torr at 25° C.

12. The solid article according to any one of the preceding clauses, wherein the one or more hydrophobic materials comprise a material selected from the group consisting of a perfume, an insect repellent, an essential oil, a functional perfume component (FPC), an aesthetic, a bioactive, a malodor counteractant, and mixtures thereof.

13. The solid article according to any one of the preceding clauses, wherein the solid article is configured to release at least 50 wt. % of the hydrophobic material when stored at 25° C. for 30 days at atmospheric pressure,

optionally wherein the solid article is configured to release at least 70 wt. % of the hydrophobic material when stored at 25° C. for 30 days at atmospheric pressure.

14. A method of making a solid article according to any one of the preceding clauses, comprising the steps:
(i) mixing an epoxidized vegetable oil with a polyfunctional carboxylic acid in a solvent;
(ii) adding the hydrophobic material and mixing until homogeneous; and
(iii) allowing the mixture to rest for at least 4 hours.

15. The method of clause 14, wherein step (i) is performed at a temperature of from 60 to 150° C.,
optionally from 80 to 120° C.

16. The method of clause 14 or 15, wherein step (i) is performed until the mixture has a viscosity of from 30 to 80 cP.

17. The method of any one of clauses 14 to 16, wherein step (ii) is performed at a temperature of from 0 to 50° C.,
optionally from 20 to 40° C.

18. The method of any one of clauses 14 to 17, wherein step (iii) is performed at a temperature of from 35 to 75° C. for a period of from 4 to 48 hours,
optionally 12 to 36 hours,
more optionally 18 to 30 hours.

19. Use of a solid article according to any one of clauses 1 to 13 for:
(a) the sustained release of a hydrophobic material selected from the group consisting of an air refresher and an insect repellant; or
(b) the sustained release of a hydrophobic material in a damp environment such as a washing machine, a dishwasher, a shower and a sink.

### DETAILED DESCRIPTION OF THE INVENTION

The invention provides a solid article for sustained release of one or more hydrophobic materials, comprising at least 5 wt. % of one or more hydrophobic materials embedded in a gel matrix,
wherein said gel matrix is formed from a chemically cross-linked polyester material.

As used herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure. When used herein, the term “substantially identical” is intended to refer to a value that is essentially identical, but for variations resulting from manufacturing tolerances. For example, the term may mean that a value varies by less than 5%, such as less than 2%, such as less than 1%, such as less than 0.5%, such as less than 0.05%, such as the value is essentially uniform.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

All measurements are performed at 25° C. unless otherwise specified.

As defined herein, a “solid article” is a product that is solid, i.e. the product does not flow and maintains its shape when stored at 25° C. The solid article is typically a self-supporting solid. For the avoidance of doubt, the solid article may be flexible and/or viscoelastic.

In some embodiments of the invention, the solid article may have a loss tangent of less than or equal to 10⁻², where the loss tangent is determined as a measure of the lost energy ratio to energy stored during cyclic deformation (tan(δ)=G″/G′), where G′ is the storage modulus and G″ is the loss modulus of the solid article.

In some embodiments of the invention, the solid article may have a storage modulus (G′) of from about 75,000 Pa to about 200,000 Pa. In some embodiments of the invention, the solid article may have a loss modulus (G″) of from about 250 Pa to about 2000 Pa.

The solid article may have a low tackiness (adhesive energy). This may be desirable from a product feel perspective, and also because a high tackiness is typically correlated with highly viscous liquids, rather than solid gels. In some embodiments of the invention, the solid article may have an adhesive energy of less than 0.2 J/m². The adhesive energy may be measured using a method as described herein.

The solid articles of the present invention may advantageously be loaded with high levels of hydrophobic material, such as up to 85 wt. % hydrophobic material. Thus, the solid article may comprise from 5 to 85 wt. % hydrophobic material, such as from 10 to 75 wt. %, from 15 to 60 wt. %, from 20 to 55 wt. %, or from 20 to 50 wt. %.

It is herein explicitly contemplated that any end point of any range defined in relation to a variable disclosed herein may be combined with any other end point from any other range defined in relation to the same variable. Thus, for the wt. % ranges for the hydrophobic material discussed above, the following ranges are also explicitly contemplated, and it is to be understood that the same principle is applied to all ranges disclosed herein for any other variable:
from 5 to 10 wt. %, from 5 to 15 wt. %, from 5 to 20 wt. %, from 5 to 25 wt. %, from 5 to 50 wt. %, from 5 to 55 wt. %, from 5 to 60 wt. %, from 5 to 75 wt. %, from 5 to 85 wt. %;
from 10 to 15 wt. %, from 10 to 20 wt. %, from 10 to 25 wt. %, from 10 to 50 wt. %, from 10 to 55 wt. %, from 10 to 60 wt. %, from 10 to 75 wt. %, from 10 to 85 wt. %;
from 15 to 20 wt. %, from 15 to 25 wt. %, from 15 to 50 wt. %, from 15 to 55 wt. %, from 15 to 60 wt. %, from 15 to 75 wt. %, from 15 to 85 wt. %;
from 20 to 25 wt. %, from 20 to 50 wt. %, from 20 to 55 wt. %, from 20 to 60 wt. %, from 20 to 75 wt. %, from 20 to 85 wt. %;
from 25 to 50 wt. %, from 25 to 55 wt. %, from 25 to 60 wt. %, from 25 to 75 wt. %, from 25 to 85 wt. %;
from 50 to 55 wt. %, from 50 to 60 wt. %, from 50 to 75 wt. %, from 50 to 85 wt. %;
from 55 to 60 wt. %, from 55 to 75 wt. %, from 55 to 85 wt. %;
from 60 to 75 wt. %, from 60 to 85 wt. %; and
from 75 to 85 wt. %.

Thus, the solid articles of the invention may include higher amounts of hydrophobic material than prior art products. In other words, the solid articles of the invention may comprise an advantageously higher amount of materials having a logP of below 6.5.

LogP refers to the log of the Octanol/Water Partition Coefficient. Unless stated otherwise, the term “logP”, as used herein, is to be understood as referring to a calculated logP (ClogP), which is calculated using the Consensus log P Computational Model, version 14.50 (Linux-based) of the ACD/Labs Percepta Batch module. The ACD/Labs' Consensus log P Computational Model is part of the deployment of ACD models on the CADMol QSAR/MolProp website.

The solid articles of the current invention, which are based on crosslinked polyester polymer gels, may incorporate and release hydrophobic materials having a very low logP of less than 3, such as from 0.01 to 3, or 0.5 to 2.8. Thus, the invention is advantageously able to overcome the problem associated with the prior art, where low logP material tend to bleed out of the gel via syneresis. Furthermore, the solid article of the invention may incorporate hydrophobic materials having a logP of greater than 3, such as from 3 to 6.5, typically from 3.5 to 5.5. Such hydrophobic materials advantageously give rise to solid articles having improved transparency, in addition to faster curing times.

Therefore, the solid articles of the invention may comprise one or more hydrophobic materials having a logP of from 0.01 to 6.5, such as 0.5 to 5.5, 1.5 to 5.5, or 2 to 5.

The solid article of the invention comprises one or more hydrophobic materials. Thus, the solid article may comprise a blend of hydrophobic materials. In such cases, the logP is the weighted average logP of the blend of hydrophobic materials.

The solid article is suitable for the release of one or more hydrophobic materials, such as volatile hydrophobic materials. Examples of volatile hydrophobic materials that are useful in the invention include perfumes, insect repellents, essential oils, functional perfume components (FPCs), aesthetic agents, bioactive agents, malodor counteractants, and mixtures thereof. Particularly suitable hydrophobic materials are perfume raw materials, especially perfume mixtures.

Suitable perfume mixtures include mixtures comprising at least one perfume raw material (PRM). Various PRMs may be used. The perfume mixture can comprise one or more of the PRMs. As used herein, a “perfume raw material” or “PRM” refers to one or more of the following ingredients: fragrant essential oils; aroma compounds; pro-perfumes; materials supplied with the fragrant essential oils, aroma compounds, including stabilizers, diluents, processing agents, and contaminants; and any material that commonly accompanies fragrant essential oils, aroma compounds.

The perfume mixture can comprise at least 20%, preferably at least 40%, even more preferably at least 70% by weight of PRMs having a logP equal or greater than 3 based on total perfume mixture weight. The perfume mixture can even comprise only PRMs having a logP equal or greater than 3. Such high logP perfume mixtures result in faster curing times, as well as greater transparency.

As mentioned earlier, the cross-linked gels of the present invention are capable of incorporating even low logP hydrophobic material. Hence, suitable perfume mixtures can comprise at least 30%, preferably at least 45%, even more preferably at least 60% or even at least 70% by weight of PRMs having a logP of less than 3 based on total perfume mixture weight. The perfume mixture can even comprise PRMs having a logP of less than 3.

The hydrophobic material is preferably volatile, especially where the hydrophobic material is a perfume or perfume mixture. In other words, typically, the one or more hydrophobic materials comprise one or more volatile hydrophobic materials. The one or more hydrophobic materials may comprise from 20 to 100 wt. % of one or more volatile hydrophobic materials, such as 40 to 100 wt. %, 60 to 100 wt. %, or 80 to 100 wt. % of one or more volatile hydrophobic materials.

The volatile hydrophobic materials may have a boiling point of less than 450° C., preferably from 60° C. to 400° C., more preferably from 75° C. to 380° C., where all boiling points are at atmospheric pressure.

In addition, or alternatively, the one or more volatile hydrophobic materials may each have a vapor pressure of at least 10⁻⁶ Torr at 25° C., such as at least 10⁻⁵ Torr at 25° C., or at least 10⁻⁴ Torr at 25° C.

At least part or all of the hydrophobic material can be non-volatile or of low volatility, having a boiling point of greater 300° C., preferably greater than 350° C. Such non-volatile or low volatility hydrophobic materials can be as eluents for perfumes and perfume mixtures.

The perfume mixture may include one or more PRM comprising reactive aldehydes, ketones and ionones. Non-limiting examples of such PRMs are Adoxal™ (2,6,10-Trimethyl-9-undecenal), Bourgeonal™ (4-t-butylbenzenepropionaldehyde), Lilestralis 33™ (2-methyl-4-t-butylphenyl)propanal), Cinnamic aldehyde, cinnamaldehyde (phenyl propenal, 3-phenyl-2-propenal), Citral, Neral (dimethyloctadienal, 3,7-dimethyl-2,6-octadien-1-al), Cyclal C™ (2,4-dimethyl-3-cyclohexen-1-carbaldehyde), Florhydral™ (3-(3-Isopropyl-phenyl)-butyraldehyde), Citronellal (3,7-dimethyl 6-octenal), Cymal (2-methyl-3-(para-isopropylphenyl)propionaldehyde), cyclamen aldehyde, Lime aldehyde (Alpha-methyl-p-isopropyl phenyl propyl aldehyde), Methyl Nonyl Acetaldehyde, aldehyde C12 MNA (2-methyl-1-undecanal), Hydroxycitronellal, citronellal hydrate (7-hydroxy-3,7-dimethyl octan-1-al), Helional™ (3-(1,3-Benzodioxol-5-yl)-2-methylpropanal); 2-Methyl-3-(3,4-methylenedioxyphenyl)propanal), Intreleven aldehyde (undec-10-en-1-al), Ligustral™ (2,4-dimethylcyclohex-3-ene-1-carbaldehyde), Trivertal™ (2,4-dimethyl-3-cyclohexene-1-carboxaldehyde), Jasmorange™ or satinaldehyde (2-methyl-3-tolylproionaldehyde, 4-dimethylbenzenepropanal), Lyral™ (4-(4-hydroxy-4-methyl pentyl)-3-cyclohexene-1-carboxaldehyde), Melonal™ (2,6-Dimethyl-5-Heptenal), Methoxy Melonal (6-methoxy-2,6-dimethylheptanal), methoxycinnamaldehyde (trans-4-methoxycinnamaldehyde), Myrac aldehyde™ (iso hexenyl tetraydrobenzaldehyde), trifernal™ ((3-methyl-4-phenyl propanal, 3-phenyl butanal), lilial (3-(4-tert-Butylphenyl)-2-methylpropanal), benzenepropanal (4-tert-butyl-alpha-methyl-hydrocinnamaldehyde), Dupical™ (muguet butanal), tricyclodecylidenebutanal (4-Tricyclo5210-2,6decylidene-8butanal), Melafleur™ (1,2,3,4,5,6,7,8-octahydro-8,8-dimethyl-2-naphthaldehyde), Methyl Octyl Acetaldehyde, aldehyde C-11 MOA (2-mehtyl deca-1-al), Onicidal™ (2,6,10-trimethyl-5,9-undecadien-1-al), Citronellyl oxyacetaldehyde, Muguet aldehyde 50™ (3,7-dimethyl-6-octenyl) oxyacetaldehyde, phenylacetaldehyde, Mefranal™ (3-methyl-5-phenyl pentanal), dimethyl tetrahydrobenzene aldehyde (2,4-dimethyl-3-cyclohexene-1-carboxaldehyde), 2-phenylproprionaldehyde, Hydrotropaldehyde (2-phenyl propionaldehyde), Canthoxal™ (para-anisyl propanal), anisylpropanal 4-methoxy-alpha-methyl benzenepropanal (2-anisylidene propanal), Cyclemone A™ (1,2,3,4,5,6,7,8-octahydro-8,8-dimethyl-2-naphthaldehyde), Precyclemone B™ (1-cyclohexene-1-carboxaldehyde), mixtures thereof, preferably the one or more non-functional perfume raw materials is selected from the group consisting of: Melonal™ (2,6-Dimethyl-5-Heptenal), Methoxy Melonal (6-methoxy-2,6-dimethylheptanal), Florhydral™ (3-(3-Isopropyl-phenyl)-butyraldehyde), iso jasmone, methyl beta naphthyl ketone, musk indanone, tonalid™ or musk tetralin, alpha-damascone, beta-damascone, delta-damascone, iso-damascone, damascenone, methyl-dihydrojasmonate, menthone, carvone, camphor, fenchone, alpha-ionone, beta-ionone, dihydro-beta-ionone, gamma-methyl ionone, alpha-methyl ionone, n-beta-methyl ionone isomer, fleuramone™ or 2-heptylcyclopentan-1-one, dihydrojasmone, cis-jasmone, iso-e-super™ or patchouli ethanone, methyl cedrylketone or methyl cedrylone, acetophenone, methyl-acetophenone, para-methoxy-acetophenone, methyl-beta-naphtyl-ketone, benzyl-acetone, benzophenone, para-hydroxy-phenyl-butanone, celery ketone or livescone™, 6-isopropyldecahydro-2-naphtone, dimethyl-octenone, freskomenthe™ or 2-butan-2-ylcyclohexan-1-one, 4-(1-ethoxyvinyl)-3,3,5,5,-tetramethyl-cyclohexanone, methyl-heptenone, 2-(2-(4-methyl-3-cyclohexen-1-yl)propyl)-cyclopentanone, 1-(p-menthen-6(2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4 (5h)-indanone, 4-damascol™ or pepper hexanone, dulcinyl™ or4-(1,3-benzodioxol-5-yl) butan-2-one, gelsone™ or ethyl 2-acetyloctanoate, hexalon™ or allyl alpha ionone, methyl cyclocitrone™ or 1-(3,5,6-trimethyl-1-cyclohex-3-enyl)ethanone, methyl-lavender-ketone™ or 3-(hydroxymethyl) nonan-2-one, orivone™ or 4-(2-methylbutan-2-yl)cyclohexan-1-one, para-tertiary-butyl-cyclohexanone, verdone™ or 2-tert-butylcyclohexan-1-one, delphone™ or 2-pentylcyclopentan-1-one, muscone, neobutenone™ or 1-(5,5-dimethyl-1-cyclohexenyl)pent-4-en-1-one, plicatone™ or octahydro-7-methyl-1,4-methanonaphthalen-6(2H)-one, veloutone™ or 2,2,5-trimethyl-5-pentylcyclopentan-1-one, 2,4,4,7-tetramethyl-oct-6-en-3-one, tetrameran™ or floral undecenone, hedione™ or methyl dihydrojasmonate, gamma undecalactone, gamma decalactone, gamma octalactone, ethylene brassylate, pentadecanolide, methyl nonyl ketone, cyclopentadecanone, 3,4,5,6-tetrahydropseudoionone, 8-hexadecenolide, dihydrojasmone, 5-cyclohexadecenone, 2-penten-1-al, 3-penten-1-al, 4-penten-1-al, (E)-2-hexen-1-al, (Z)-2-hexen-1-al, 3-hexen-1-al, 4-hexen-1-al, 2-hepten-1-al, 3-hepten-1-al, 4-hepten-1-al, 2-octen-1-al, 3-octen-1-al, 4-octen-1-al, 2-nonen-1-al, 3-nonen-1-al, 4-nonen-1-al, 5-nonen-1-al, 2-decen-1-al, 3-decen-1-al, 4-decen-1-al, 5-decen-1-al, 2-undecen-1-al, 3-undecen-1-al, 4-undecen-1-al, 5-undecen-1-al, 2-dodecen-1-al, 3-dodecen-1-al, 4-dodecen-1-al, 5-dodecen-al-1, 2-tridecen-1-al, 3-tridecen-1-al, 4-tridecen-1-al, 2-tetradecen-1-al, 3-tetradecen-1-al, 4-tetradecen-1-al, 5-tetradecen-1-al, and mixtures thereof.

As discussed herein, the perfume mixture may include one or more reactive aldehydes that contribute to scent character and neutralize malodors in vapor and/or liquid phase via chemical reactions. Aldehydes that are partially reactive or volatile may be considered a reactive aldehyde as used herein. Reactive aldehydes may react with amine-based odors, following the path of Schiff-base formation. Reactive aldehydes may also react with sulfur-based odors, forming thiol acetals, hemi thiolacetals, and thiol esters in vapor and/or liquid phase. It may be desirable for these vapor and/or liquid phase reactive aldehydes to have virtually no negative impact on the desired perfume character, color or stability of a product. Examples of suitable reactive aldehydes include the aldehydes disclosed above.

The perfume mixture may include one or more of the following perfume raw materials: cyclic ethylene dodecanedioate, 4-tertiary butyl cyclohexyl acetate or vertenex™, allyl amyl glycolate, allyl caproate, allyl cyclohexane propionate, allyl heptanoate, amber xtreme, ambrox, isoamyl acetate, isoamyl propionate, anethole usp, benzyl propionate, cis-3-hexen-1-ol, beta naphthol methyl ether or nerolin, caramel furanone, caryophyllene extra, cinnamalva™ or Cinnamyl Nitrile, cinnamyl acetate, cinnamyl nitrile, cis-3-hexenyl butyrate, cis-3-hexenyl acetate, cis-3-hexenyl alpha methyl butyrate, cis-6-nonen-1-ol, citrathal or citral diethyl acetal, citronellol, citronellyl acetate, citronellyl butyrate, clonal or dodecane nitrile, coranol or 2,2-dimethyl cyclohexanepropanol, coumarin, cumin nitrile, cuminic alcohol, tricyclodecenyl isobutirate or cyclabute, cyclohexyl ethyl acetate, dihydromyrcenol, dimethyl anthranilate, dimethyl benzyl carbinyl acetate, dimethyl-2 6-heptan-2-ol or freesiol, sandal pentenol or ebanol, ethyl-2-methyl pentanoate, ethyl acetoacetate, ethyl linalool, ethyl maltol, ethyl phenyl glycidate, ethyl vanillin, ethyl-2-methyl butyrate, eucalyptol, eugenol, flor acetate, ozone propanal or floralozone, fructalate™ or raspberry dicarboxylate, geraniol or trans-3,7-dimethyl-2,7-octadien-1-ol, grisalva™ or amber furan, habanolide™ or (E)-12-musk decenone, helvetolide™ or musk propanoate, hexyl acetate, hexyl-2-methyl butyrate, indocolore™ or 1-phenylvinyl acetate, iso bornyl acetate, iso eugenyl acetate, isoamyl butyrate, isoeugenol, koumalactone™ or dihydromint lactone, laevo trisandol or sandranol, lemonile™ or homogeranyl nitrile, levistamel™ or mesitene lactone, linalool, linalyl acetate, linalyl iso butyrate, lymolene or dihydromyrcenol, menthol, methyl dioxolan or fructone™M, methyl iso butenyl tetrahydro pyran, methyl pamplemousse™ or grapefruit acetal, methyl phenyl carbinyl acetate or styrallyl acetate, methyl salicylate, montaverdi™ or green cyclopropionate, mugetanol™ or muguet ethanol, neocaspirene, neofolione or melon nonenoate nerolidol, orange terpenes, orcinyl-3 or 3-methoxy-5-methylphenol, oxane™ or cis-galbanum oxathiane, para cresyl methyl ether or para methyl anisole, patchouli, phenyl ethyl alcohol, phenyl ethyl dimethyl carbinol, polysantol™ or santol pentenol, prenyl acetate, sauvignone™ or 5-mercapto-5-methyl-3-hexanone, sclareolate™ or clary propionate, shisolia, strawberiff™ or 2-methyl-2-pentenoic acid, terpinolene or 4-isopropylidene-1-methylcyclohexene, tetrahydro muguol™ or citrus ocimenol, thesaron™ (1R,6S)-2,2,6-Trimethyl-cyclohexanecarboxylic acid ethyl ester, tobacarol™ or 5-tetramethyl oxatricyclododecane, undecavertol™ or violet decenol, verdox™ or green acetate, verdural B™ or (Z)-3-hexen-1-yl isobutyrate, violettyne™ or violet dienyne, violiff™ or violet methyl carbonate, and mixtures thereof.

The perfume mixture may contain functional perfume components (“FPCs”). FPCs are a class of perfume raw materials with evaporation properties that are similar to traditional organic solvents or volatile organic compounds (“VOCs”). “VOCs”, as used herein, means volatile organic compounds that have a vapor pressure of greater than 0.2 mm Hg measured at 20° C. and aid in perfume evaporation. Exemplary VOCs include the following organic solvents: dipropylene glycol methyl ether (“DPM”), dimethyl adipate, 3-methoxy-3-methyl-1-butanol (“MMB”), volatile silicone oil, and dipropylene glycol esters of methyl, ethyl, propyl, butyl, ethylene glycol methyl ether, ethylene glycol ethyl ether, diethylene glycol methyl ether, diethylene glycol ethyl ether, or any VOC under the tradename of Dowanol™ glycol ether. VOCs are commonly used at levels greater than 20% in a fluid composition to aid in perfume evaporation.

The FPCs aid in the evaporation of perfume materials and may provide a hedonic, fragrance benefit. FPCs may be used in relatively large concentrations without negatively impacting perfume character of the overall composition. As such, the fluid composition may be substantially free of VOCs, meaning it has no more than 18%, alternatively no more than 6%, alternatively no more than 5%, alternatively no more than 1%, alternatively no more than 0.5%, by weight of the composition, of VOCs. The fluid composition may be free of VOCs.

Perfume materials that are suitable for use as a FPC can also be defined using odor detection threshold (“ODT”) and non-polarizing scent character for a given perfume character scent camp.

FPCs may have an ODT from greater than 1.0 parts per billion (“ppb”), alternatively greater than 5.0 ppb, alternatively greater than 10.0 ppb, alternatively greater than 20.0 ppb, alternatively greater than 30.0 ppb, alternatively greater than 0.1 parts per million.

FPCs may be volatile, low boiling point (B.P.) perfume materials. Exemplary FPC include iso-nonyl acetate, d-limonene, 1-methyl-4-isopropenyl-1-cyclohexene, benzyl acetate, benzyl benzoate, isopropyl myristate, diethyl phthalate and mixtures thereof.

The total amount of FPCs in the perfume mixture may be greater than 10%, alternatively greater than 20%, alternatively greater than 30%, alternatively greater than 50%, alternatively greater than 60%, alternatively greater than 70%, alternatively greater than 80%, alternatively from 30% to 100%, alternatively from 50% to 100%, alternatively from 60% to 100%, alternatively from 70% to 100%, alternatively from 80% to 100%, alternatively from 85% to 100%, alternatively from 90% to 100%, by weight of the perfume mixture. The perfume mixture may consist entirely of FPCs (i.e. 100 wt. %).

Preferably, the hydrophobic material is a liquid under ambient conditions (such as from 5° C. to 25° C.).

Suitable insect repellents include any typical insect and/or moth repellents such as citronellal, citral, geraniol, citridiol, N,N-diethyl-meta-toluamide, Rotundial, 8-acetoxycarvotanacenone, peppermint oil, cinnamon oil, spearmint oil, lavender oil, clove oil, lemongrass oil, garlic oil, cornmint oil, rosemary oil, soybean oil, thyme oil, geranium oil, and combinations thereof. Other examples of insect and/or moth repellent for use herein are disclosed in U.S. Pat. Nos. 4,449,987, 4,693,890, 4,696,676, 4,933,371, 5,030,660, 5,196,200, “Semio Activity of Flavour and Fragrance molecules on various Insect Species”, B. D. Mookherjee et al., published in Bioactive Volatile Compounds from Plants, ASC Symposium Series 525, R. Teranishi, R. G. Buttery, and H. Sugisawa, 1993, pp. 35-48.

The solid articles of the invention may include one or more sensates, which may be added to improve one or more sensory properties of the solid articles (e.g. aroma). Suitable sensates include menthol (L, D, racemic), eucalyptol and eucalyptus oil, peppermint oils, cornmint or arvensis 15 mint oils, spearmint oils, carvone, clove oils, cinnamic aldehyde and cinnamon derivatives, aliphatic carboxamides, ketals, cyclohexyl derivatives, mono-menthyl succinated and mixtures thereof. Some examples are: WS-3 available as ISE 3000 and WS-23 available as ISE 1000 from Qaroma, Inc. MGA available from Symrise, TK10, Coolact available from LIPO Chemicals of Paterson, N.J., and Physcool™.

The solid articles of the invention may include one or more aesthetics, which may be added to enhance the appearance of the gel. Examples of suitable aesthetics include colorants such as dyes or pigments, and other aesthetic materials such as particles that may be suspended within the solid articles (which particles may have different shapes and sizes). Non-limiting examples of colorants are Rhodamine, Fluorescein, Phathalocyanine, alumina and mixtures thereof. Non-limiting examples of particles that may be suspended within the solid articles (with different shapes and sizes) include glitter (and glitter-type materials), epoxy coated metalised aluminium polyethylene terephthalate, polyester beads, candelilla beads, silicates and mixtures thereof. Such aesthetic materials are available from Glittergo Limited, Impact colors and CQV Co. Ltd.

The one or more hydrophobic materials are embedded in a gel matrix, which may be referred to herein as a “gel”, “polymer gel”, or “polymer gel matrix”. The gel matrix is formed from a chemically cross-linked polyester material. As used herein, “embedded” means that the one or more hydrophobic materials are present physically within the gel matrix but are not chemically bonded to the gel matrix. Any type of interaction between the hydrophobic materials and polymer gel matrix may be present within the scope of the current invention, provided that the interaction does not prevent release of the hydrophobic material over time. Non-limiting examples of interactions that may be present between the gel matrix and hydrophobic material include hydrogen bonding, dipole-based interactions and Van der Waals interactions.

The solid article of the invention may be tuned to release a desired amount of a hydrophobic material over a specific time period. The release of the hydrophobic material may be controlled by changing the identity of the hydrophobic material and/or the gel matrix, and more specifically by changing the nature of the interactions between the hydrophobic material and the gel matrix (e.g.

by changing the hydrophobicity/hydrophilicity of the hydrophobic material and/or gel matrix), as well as the mesh size of the gel matrix (e.g. by controlling the degree of crosslinking). Thus, it is possible to prepare solid articles that will release a hydrophobic material very quickly (e.g. within a period of a one to two weeks), or over a longer time period (e.g. at least eight weeks or at least twelve weeks).

In some embodiments of the invention, the solid article may be configured to release at least 5 wt. % of the hydrophobic material when stored at 25° C. for 30 days at atmospheric pressure. In some embodiments of the invention, the solid article may be configured to release at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, at least 65 wt. %, at least 70 wt. %, at least 75 wt. %, or at least 80 wt. %, of the hydrophobic material when stored at 25° C. for 30 days at atmospheric pressure. In specific embodiments of the invention, the solid article may be configured to release at least 50 wt. %, or at least 70 wt. % of the hydrophobic material when stored at 25° C. for 30 days at atmospheric pressure.

The polymer gel matrix is formed from a chemically crosslinked polyester material. Any suitable chemically crosslinked polyester material may be used in the current invention. Exemplary chemically crosslinked polyester materials may be prepared from the starting materials discussed hereinbelow.

The chemically crosslinked polyester material may be prepared from an epoxidized oil (e.g. epoxidized vegetable oil) having at least two epoxy groups, and a chemical crosslinker such as a polyfunctional carboxylic acid. The epoxidized oil may have from 2 to 8, such as 2 to 6, epoxy groups per molecule. Preferably, the epoxidized oil has an oxirane oxygen content of at least 5%, such as at least 5.5%, e.g. at least 6%. In some embodiments the epoxidized oil has an oxirane oxygen content of from 5% to 10%, such as from 5.5% to 9%, e.g. from 6% to 8.5%. As will be appreciated by a person skilled in the art, the greater the degree of epoxidation of the epoxidized oil, the more sites are available for crosslinking during gel formation. Thus, oxirane oxygen content may loosely correlate with the degree of crosslinking, provided sufficient crosslinker is present. The greater the degree of crosslinking within a gel matrix, the slower the release of hydrophobic material from the gel matrix.

Another analytical measure that relates to the degree of crosslinking is the correlation length of the polymer gel matrix. In general, for polymer gels based on the same starting materials (e.g. the same epoxidized oil and multivalent carboxylic acid), a lower correlation length indicates a higher degree of crosslinking. However, correlation lengths may be less comparable between polymers that are prepared from different epoxidized oils and/or different multivalent carboxylic acids. Nevertheless, in general, the solid articles of the invention may have a correlation length as measured by Small-angle X-ray scattering (SAXS) of less than or equal to 4 nm, such as from about 0.05 nm to 4 nm, preferably from 0.08 nm to 2 nm, even more preferably from about 0.1 to 0.5 nm.

The chemically crosslinked polyester material may advantageously be prepared from the starting materials without the need for any catalyst or additive, and in any solvent that does not interfere in the reaction and in which any solid materials are soluble (such as non-nucleophilic/non-alcoholic solvents).

Examples of suitable epoxidized oils include epoxidized fatty acid and/or triglyceride materials containing at least two epoxy groups per molecule, though a skilled person will realise that other epoxy-containing oils may also be used. Specific examples of suitable oils include epoxidized linseed oil, epoxidized soybean oil, epoxidized castor oil, epoxidized rapeseed oil, epoxidized vernonia oil, vernonia oil, epoxidized corn oil, epoxidized cottonseed oil, epoxidized canola oil, epoxidized grape seed oil, epoxidized poppy seed oil, epoxidized tung oil, epoxidized sunflower oil, epoxidized safflower oil, epoxidized walnut oil. Preferred epoxidized oils include epoxidized linseed oil, epoxidized soybean oil, and epoxidized castor oil. A particularly preferred epoxidized oil is epoxidized soybean oil.

Exemplary structures of certain epoxidized oils are provided below. These structures depict fully epoxidized oils having no carbon-carbon double bonds (alkene groups). However, a person skilled in the art will appreciate that any epoxidized oil having at least two epoxy groups may be used in the current invention, such as partially epoxidized variants of the below structures.

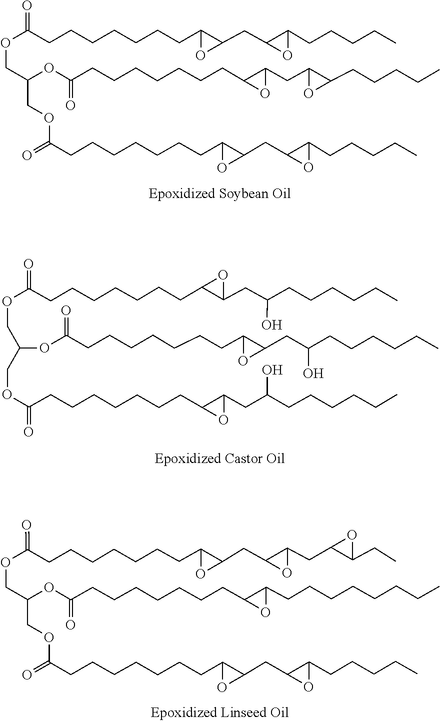 [SMILES: CCCCCC1OC1CC1OC1CCCCCCCC(=O)OCC(COC(=O)CCCCCCCC1OC1CC1OC1CCCCC)OC(=O)CCCCCCCC1OC1CC1OC1CCCCC ; CCCCCCC(O)CC1OC1CCCCCCCC(=O)OCC(COC(=O)CCCCCCCC1OC1CC(O)CCCCCC)OC(=O)CCCCCCCC1OC1CC(O)CCCCCC ; CCCCCCCCC1OC1CCCCCCCC(=O)OC(COC(=O)CCCCCCCC1OC1CC1OC1CCCCC)COC(=O)CCCCCCCC1OC1CC1OC1CC1OC1CC]

Any suitable crosslinking agent capable of crosslinking the epoxidized oil to form a polyester may be used, such as a polyfunctional carboxylic acid (e.g. a carboxylic acid having two or three acid groups, though carboxylic acids having more than three acid groups may also be used). Any polyfunctional carboxylic acid that is soluble in the solvent of choice may be used, with natural carboxylic acids being preferred from a sustainability and cost viewpoint. Specific polyfunctional acids that may be used include citric acid, maleic acid, itaconic acid, fumaric acid, succinic acid, methylsuccinic acid, malic acid, malonic acid, phthalic acid, tartaric acid, citraconic acid, and furan dicarboxylic acid, such as citric acid, maleic acid, itaconic acid, fumaric acid, succinic acid, methylsuccinic acid, malic acid, tartaric acid, citraconic acid, and furan dicarboxylic acid. A particularly preferred polyfunctional acid that may be used is citric acid.

Examples of suitable solvents include water, acetone and toluene, with water being particularly preferred from a point of view of environmental impact and cost of manufacture.

As mentioned above, any solid materials used in the preparation of the solid articles of the invention must be soluble in the solvent used. Thus, the crosslinker (e.g. polyfunctional carboxylic acid) must be soluble in the solvent used. Preferably, the crosslinker (e.g. polyfunctional carboxylic acid) has a solubility in a non-nucleophilic/non-alcoholic solvent of at least 100 g/L, such as at least 200 g/L, at least 300 g/L or at least 400 g/L. More preferably, the solvent is water.

Nevertheless, a skilled person will understand that solid articles may be prepared from materials that have a low solubility in water, such as a solubility of 0.1 g/L in water. In such cases, an alternative solvent may be used, e.g. toluene, and the materials used to prepare the solid article have a higher solubility in said alternative solvent, such as at least 50 g/L, or at least 100 g/L, at least 200 g/L, at least 300 g/L or at least 400 g/L.

By way of example, phthalic acid, citric acid and maleic acid may be used to form gels with epoxidized soybean oil using water as a solvent. However, succinic acid and trimesic acid, which are far less soluble in water, are unable to form a gel when water is used as a solvent. Nevertheless, a person skilled in the art will understand that these acids will have increased solubility in other solvents such as toluene, and appropriate polymer gels may be prepared from these acids in such other solvents.

As will be appreciated by a person skilled in the art, an appropriate polymer gel may be prepared from mixtures of starting materials and solvents, i.e. mixtures of epoxidized oils and/or mixtures of polyfunctional carboxylic acids and/or mixtures of solvents.

Advantageously, the solid articles of the current invention have improved biodisintegration as compared to prior art solid articles. For example, the solid articles may have a biodisintegration of at least 25% as measured by the standard method UNE-EN-ISO-20200:2016. In some embodiments of the invention, the solid article may have a biodisintegration of at least 35%, at least 40%, at least 50%, at least 55%, at least 60%, at least 65%, at least 70%, at least 75%, at least 80% or at least 85% as measured by the standard method UNE-EN-ISO-20200:2016. As used herein, “biodisintegration” of the solid articles refers to the biodisintegration as measured by UNE-EN-ISO-20200:2016, conducted on a solid article that has exhausted its loading of hydrophobic material. This is because evaporation of hydrophobic material during a biodisintegration test will provide an inaccurate measurement.

Advantageously, the solid articles of the current invention may have improved transparency, and therefore improved aesthetic value. This advantageously increases consumer appeal, resulting in improved commercial performance.

The solid articles of the current invention may have any appropriate thermal conductivity. For example, the solid articles may have a thermal conductivity of from about 0.17 to 0.2 W/m·K, such as about 0.18 to 0.19 W/m·K. Without being bound by theory, it is believed that solid articles having a thermal conductivity within this range have a more consistent release of hydrophobic material over time, as compared to articles with other thermal conductivities. Thermal conductivity of the solid article may be influenced by the degree crosslinking in the gel matrix, which itself may be influenced by the degree of epoxidation of an epoxidized oil starting material.

The solid article of the invention may have any appropriate haze value. Nevertheless, the solid article may have a haze of less than 80%, such as less than 50%, or less than 20%, since a lower haze results in an advantageously more aesthetically pleasing product.

The solid article of the invention may have any suitable shape and size. Since the solid articles of the invention release the hydrophobic material from the surface of the solid article (e.g. by evaporation), the surface area of the solid article will affect the release rate of the hydrophobic material. For instance, for the same mass of the solid article, thin sheets result in faster hydrophobic material release and lower product lifetime than spheres. For typical applications, the solid article may have a surface area of less than about 150 cm², such as from about 0.5 cm² to about 100 cm², e.g. from about 1 cm² to about 60 cm². For typical applications, the solid article may have a volume of from about 0.2 cm³ to about 25 cm³, such as about 0.5 cm³ to about 15 cm³. Nevertheless a person skilled in the art will appreciate that solid articles of other sizes may be useful for certain applications.

The lifetime of a product will be influenced by the release rate of hydrophobic material, and the total amount of hydrophobic material present. Since the release rate will depend on the surface area, and the total amount of hydrophobic material present may depend on the volume of the product (for any given concentration of hydrophobic material), this principle may be understood by reference to surface area: volume ratio. In order to maintain a desirable balance between product lifetime, release rate and overall dimensions, for typical applications it may be desirable for the solid article to have a surface area: volume ratio of from about 2 cm⁻¹ to about 15 cm⁻¹, such as about 4 cm⁻¹ to about 10 cm⁻¹. Nevertheless, a skilled person will appreciate that the solid articles having other surface area:volume ratios may be useful in certain applications.

As will be appreciated by a person skilled in the art, one or more solid articles may be used to provide a desired release of hydrophobic material in an environment. For example, if a very high release rate is desired then one may use multiple solid articles each having a relatively low volume, but with a very high surface area for said volume (i.e. a high surface area: volume ratio), as compared to using a single solid article. In these cases, the lifetime of the solid articles may be lower, on account of a higher evaporation rate resulting from the increased surface area. A skilled person will appreciate that the opposite situation may also apply, and a solid article having a low surface area: volume ratio may be used in a situation where a lower release rate is desired.

The surface area can be measured by creating a 3D model of the solid article using CAD software, and using the CAD software to calculate the surface area. Any suitable CAD software can be used, such as AutoCad® 2013.

The solid article of the invention may be useful in a wide range of applications, as described herein. A skilled person will be aware of many possible scenarios where release of a hydrophobic material such as an air freshening composition/perfume may be desirable. Non-limiting examples include the following.
The interior of small bags/pouches, such as sports bags, purse, backpacks, beauty-cases, suitcases, and luggage.
The interior of enclosed spaces, such as rooms (e.g. bathrooms) and cars.
Wet household spaces/damp environments (e.g. showers, toilets, washing machines, dishwashers, sinks, and drains). The solid articles of the invention may be added directly to the wet space, such as directly into the drum of a washing machine in order to slowly release the hydrophobic material (e.g. a perfume or a malodor prevention chemical) over time.
The interior of household furniture, such as drawers, closets, shoe cabinets.
The interior of trash cans and bins.
Pet housing or pet-related items such as cat litterboxes, dog baskets and cages.
On the go freshness, such as wearable items (e.g. pins to attach to clothes), and rub-on sticks.
Spatial insect (e.g. mosquito) repellent with essential oils or other volatile insect-repelling compounds.

Therefore, the invention also provides a method of delivering a hydrophobic material as described herein to an environment, the method comprising a step of placing a solid article as described herein into an environment disclosed in the list above (e.g. into the interior of an enclosed space), and allowing the hydrophobic material to evaporate from the solid article. The invention also provides the use of a solid article as described herein for the sustained release of a hydrophobic material as described herein and/or for the sustained release of a hydrophobic material as described herein to an environment disclosed herein, such as in the list above.

The invention also provides a method for making the solid articles. Thus, the invention provides a method of making a solid article as disclosed herein, comprising the steps:
(i) mixing an epoxidized vegetable oil with a polyfunctional carboxylic acid in a solvent;
(ii) adding the hydrophobic material and mixing until homogeneous; and
(iii) allowing the mixture to rest for at least 4 hours.

Step (i) may typically be performed at elevated temperature, such as a temperature of from 60 to 150° C., preferably from 80 to 120° C. Step (i) may typically be performed until the reaction mixture has reached a specific viscosity that indicates partial polymerization of the epoxidized vegetable oil and polyfunctional carboxylic acid, such as a viscosity of from 30 to 80 cP (such as 40 to 60 cP) at the reaction temperature.

Step (ii) may typically be performed at a lower temperature than step (i), so as to slow down the polymerization reaction whilst the hydrophobic material is incorporated into the mixture. Thus, step (ii) may be performed at a temperature of from 0 to 50° C., preferably from 20 to 40° C.

Step (iii) represents a curing step, and may be performed at any appropriate temperature and for any appropriate duration so as to allow for appropriate curing of the mixture to provide a polymer gel incorporating the hydrophobic material. Thus, step (iii) may be performed at a temperature of from 35 to 75° C. for a period of from 4 to 48 hours, such as from 12 to 36 hours, or from 18 to 30 hours. Typically, step (iii) may be performed by placing the mixture from step (ii) in a mold (e.g. a silicon mold) of a desired size and shape, and covering the mold to prevent evaporation of the hydrophobic material.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

The invention is illustrated by the below Examples, which are not to be construed as limitative.

### EXAMPLES

### Materials

Materials were obtained from the following suppliers.

Soybean epoxidized oil (oxirane oxygen content of 6.6%) was obtained from Xiamen through CFT (China).

Castor epoxidized oil (oxirane oxygen content of 6.8%) was obtained from Specific Polymers (France).

Linseed epoxidized oil (oxirane oxygen content of 8%) was obtained from Traquisa (Spain). Citric acid, maleic acid and phthalic acid were obtained from Sigma-Aldrich (USA).

### Characterisation Methods

### Method for Determining Oxirane Oxygen Content:

The oxirane oxygen content (OOC, %) in epoxidized vegetable oils is determined via the standard method ISO 3001:1999(E).

### Method for Determining Bio-Disintegration:

The degree of disintegration of a solid article disk is determined via the standard method UNE-EN-ISO-20200:2016 under simulated composting conditions in a laboratory-scale test.

### Method for Determining LogP:

LogP was determined as a calculated logP (ClogP), which was calculated using the Consensus log P Computational Model, version 14.50 (Linux-based) of the ACD/Labs Percepta Batch module. The ACD/Labs' Consensus log P Computational Model is part of the deployment of ACD models on the CADMol QSAR/MolProp website.

### Viscosity Determination:

Viscosity is determined using a Brookfield DV2T viscosimeter (DV2TLVTJO model) with geometry LV-04 (64) calibrated with viscosity reference standard S600, Lot number 2193007 (Paragon Scientific Ltd) at the required temperature. Data points were taken as an average of the viscosity measured during 1 min with the viscosimeter operating at 100 rpm.

### Method for Determining the Viscoelastic Moduli of a Gel Composition:

Viscoelastic properties (the storage (G′) and the loss (G″) modulus) are measured using a controlled strain rheometer (such as an ARES GII from TA Instrument, Inc., or equivalent), used in torsion mode. Rectangular solid articles conform to the dimensions of 12.5±2 mm length, 11.5±0.5 mm width and 3.3±0.5 mm thickness, are used. Small amplitude oscillatory torsional tests are performed over the range 100-0.03 rad/s at 25° C. within the linear viscoelastic regime, which is previously determined by applying oscillatory strain sweeps at 0.1Hz from 0.01 to 1% of total deformation. Viscoelastic moduli are obtained as an average of 2 repetitions, taking 4 points per decade logarithmically distributed over the frequency range.

### Method for Determining the Gel Structural Strength:

The gels structural strength at rest is expressed, in these gel compositions, by use of the minimum value of the loss tangent vs frequency plot. The loss tangent is determined as a measure of the lost energy ratio to energy stored during cyclic deformation (tan(δ)=G″/G′), using a controlled strain rheometer (such as an ARES GII from TA Instrument, Inc., or equivalent), in torsion mode. Rectangular solid articles with dimensions of 12.5±2 mm length, 11.5±0.5 mm width and 3.3±0.5 mm thickness, are used. Small amplitude oscillatory torsional tests are performed in the range 100-0.03 rad/s at 25° C. within the linear viscoelastic regime.

### Method for Determining the Adhesive Energy of a Gel Composition:

The adhesive energy (tackiness) is determined using a controlled-stress rheometer (such as MARS rheometer, Thermo Haake, Germany) using smooth steel plate-plate geometries (35 mm), at 25° C. The solid article disk with dimensions of 35±2 mm diameter and 3.3±0.5 mm thickness is exposed to an initial normal force of 3N and a de-bonding speed (Vd) of 0.1 mm/s. The contact time between gel and surface was stablished in 60 s. The required normal force for debonding and the subsequent measuring gap was collected as a function of time. For each system, adhesion energy (J/m²) is calculated as the area below the experimentally obtained force-displacement (J) curve normalized to the surface area of the probe (m²).

$${E}_{ad} = {h}_{0} {{\int }^{0}}_{{ε}_{max}} σ ( ε ) d ε$$

Where the values of initial adhesive thickness (h0), the normal stress (σ) and the strain (ε) are directly obtained from the probe tack test.

### Method for Determining the Hydrophobic Material Release:

Hydrophobic material release of a solid article disk (40±2 mm diameter and 3.3±0.5 mm thickness) is determined by measuring the weight loss of the disk at 25±2° C. and at 60% relative humidity.

### Method for Determining the Thermal Conductivity:

Thermal conductivity is measured via standard test method ASTM D5930-17 (Thermal Conductivity of Plastics by Means of a Transient Line-Source Technique) using Xiatech TC3000E transient hotwire thermal conductivity meter Instrument. Measurements were carried out at a temperature of 24±2° C.

### Method for Determining the Microstructure/Correlation Length (via SAXS):

Sample preparation: A small (˜1 mm×˜1 mm×˜3 mm) segment of gel is cut using a scalpel and it is placed into a demountable cell with Kapton film windows giving a sample thickness of 1 mm. SAXS measurements are performed using a HECUS, S3-MICRO Kratky-type camera equipped with a position sensitive, 50M OED detector comprising of 1024 channels, 54 μm in width. An ultra-brilliant point microfocus X-ray source (GENIX-Fox 3D, Xenocs, Grenoble) provides Cu Kα radiation with a wavelength, λ, of 1.542 Å at a maximum power of 50 W. A sample-to-detector distance of 281 mm allows for a measurable q-range between 0.01 and 0.54 Å−1 (where q, the scattering vector, is given by q=4π/λ sin θ, and 2θ is the scattering angle). The S3-MICRO camera is calibrated using silver behenate (d=58.38 Å) and kept under vacuum to reduce scattering from air. Measurements are performed at a temperature of 25° C. and controlled by a Peltier element with an accuracy of 0.1° C. Raw scattering data is corrected for the scattering of the cell and unreacted pure castor oil using a relative transmission factor.

Data analysis: The intensity of scattered radiation, I(Q), for a typical gel has a Q-dependence following a Lorentzian form (equation 1.0) (Hammouda, B. Insight into Clustering in Poly(ethylene oxide) Solutions. Macromolecules 2004. 37, 6932-6937):

$$I ( Q ) = \frac{I ( 0 )}{1 + {( Q ξ )}^{2}}$$

wherein,
I (Q) is intensity of scattered irradiation
I (0) is the scattering length intensity at q=0
ξ is the correlation length

This describes scattering from a polymer where the polymer chains are considered as a “blob” of size ξ. A plot of 1/I(Q) vs. Q2 will yield a straight line in the low q region with a intercept of 1/I(0) and slope of ξ2/I(0) from which the correlation length can be obtained.

### Method for Determining Haze:

Haze (%) is measured using a Spectrophotomer (such as HunterLab UltraScan Vis, with a wavelength range from 360 to 780 nanometers) in Total Transmittance mode (TTRAN) using Illuminant D65/10 and an area view of 1 inch. The experiment is conducted at an environmental temperature of 20 degrees Celsius. The homogeneous sample, without air bubbles or cracks, with a diameter of 40±2 mm and a thickness of 3.3±0.5 mm is placed against the transmission port using a transmission clamp or similar device to maintain the sample against the transmission port. A transmission haze measurement is a ratio of the diffuse light to the total light transmitted by the sample, calculated as follows:

$$Haze is expressed as \% . Haze = \frac{{Y}_{Diffuse Transmission}}{{Y}_{Total Transmission}} \times 100.$$

### General Synthetic Method 1

Polyester polymer gels were prepared according to the following general method.

First, an acid solution is prepared in a beaker by mixing the required amounts of acid and the solvent (see Table 1 below) at 300 rpm with a magnetic stirrer for 5 min at 30° C. Then, epoxidized oil is added, heated to 100° C. and mixed at 300 rpm. Temperature is maintained until the reaction mixture has a viscosity of about 50±10 cP. Then, the system is cooled down to 35° C. using a cold-water bath and hydrophobic material is added and mixed till it is completely homogeneous. Then, the blend is poured in silicon molds having the desired shape (see characterization methods above for the size of the solid articles used for each method). The silicon molds are then covered to prevent evaporation of the hydrophobic material, and kept at 50° C. in an oven for 24 hours to cure the blend.

An exemplary reaction scheme corresponding to the preparation of a polyester polymer gel from epoxidized soybean oil and citric acid is provided below.

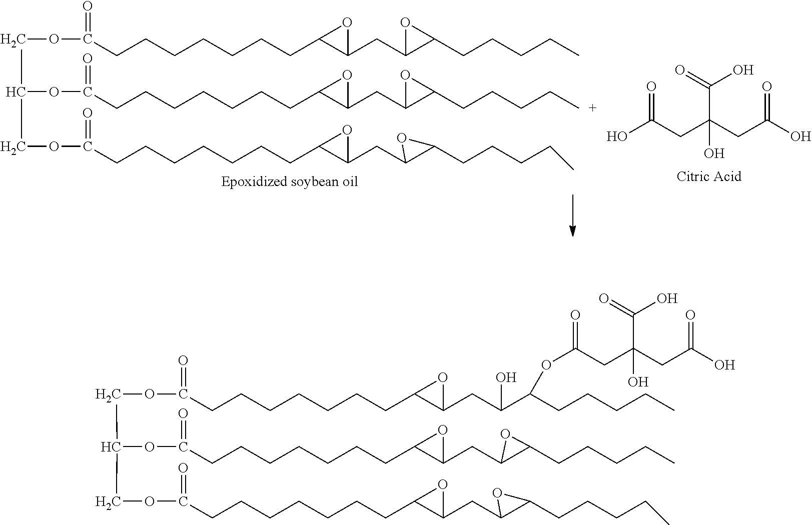

As will be appreciated by a person skilled in the art, the presence of other carboxylic acid groups on the citric acid allows it to crosslink multiple epoxidized soybean oil molecules. Furthermore, the presence of other epoxy groups on the epoxidized soybean oil molecule allows further reactions with other molecules of acid to form a crosslinked polymer gel network.

Analogous reactions will occur for other epoxidized oils, e.g. epoxidized castor and linseed oils.

### Examples 1-9

Solid articles according to the invention were prepared using Hydrophobic Materials A-D, the properties of which are listed in Table 1 below.

TABLE 1

| | Hydrophobic | Hydrophobic | Hydrophobic | Hydrophobic |
|---|---|---|---|---|
| Hydrophobic | material A | material B | material C | material D |
| material properties | (wt. %) | (wt. %) | (wt. %) | (wt. %) |
| PRMs % comprising | 18.05 | 28.00 | 21.50 | 27.42 |
| alcohols | | | | |
| PRMs % comprising | 27.60 | 20.00 | 27.00 | 12.93 |
| aldehydes | | | | |
| PRMs % comprising | 8.16 | 16.00 | 15.00 | 4.60 |
| ketones | | | | |
| PRMs % comprising | 42.80 | 36.00 | 18.00 | 18.16 |
| esters | | | | |
| PRMs % comprising | 3.39 | 0 | 18.50 | 36.89 |
| other functionalities | | | | |
| PRMs % comprising a | 27.10 | 68.00 | 0 | 1.45 |
| logP <2 | | | | |
| PRMs % comprising a | 30.03 | 32.00 | 0 | 17.64 |
| 2 < logP < 3 | | | | |
| PRMs % comprising a | 22.72 | 0 | 0 | 47.45 |
| 3 < logP < 4 | | | | |
| PRMs % comprising a | 5.43 | 0 | 30.50 | 26.46 |
| 4 < logP < 5 | | | | |
| PRMs % comprising a | 14.72 | 0 | 69.50 | 7.00 |
| logP >5 | | | | |
| PRMs % comprising a | 0.16 | 0 | 30.00 | 1.27 |
| Vap. Pres. (25° C.) <10⁻⁴ | | | | |
| PRMs % comprising a | 0.75 | 4.00 | 58.00 | 8.59 |
| 10⁻⁴ < Vap. Pres. | | | | |
| (25° C.) < 10⁻³ | | | | |
| PRMs % comprising a | 8.69 | 12.00 | 3.00 | 16.15 |
| 10⁻³ < Vap. Pres. | | | | |
| (25° C.) < 10⁻² | | | | |
| PRMs % comprising a | 42.14 | 44.00 | 9.00 | 26.07 |
| 10⁻² < Vap. Pres. | | | | |
| (25° C.) < 10⁻¹ | | | | |
| PRMs % comprising a | 48.26 | 40.00 | 0 | 47.92 |
| 10⁻¹ < Vap. Pres. | | | | |
| (25° C.) < 1 | | | | |

Solid articles according to the invention were prepared according to General Synthetic Method 1 using the materials listed in Table 2 below.

TABLE 2

| Raw Materials | Example No. | | | | | | | | |
|---|---|---|---|---|---|---|---|---|---|
| (wt. %) | 1 | 2 | 3 | 4 | 5 | 6 | 7 | 8 | 9 |
| Epoxidized | 67.2 | | | 64.5 | 64.2 | 67.2 | 67.2 | 39.8 | 63.4 |
| soybean oil | | | | | | | | | |
| Epoxidized | | 68.3 | | | | | | | |
| castor oil | | | | | | | | | |
| Epoxidized | | | 65 | | | | | | |
| linseed oil | | | | | | | | | |
| Citric acid | 7.1 | 6.5 | 8.3 | | | 7.1 | 7.1 | 6.8 | 8.3 |
| Maleic acid | | | | 8.6 | | | | | |
| Phthalic acid | | | | | 8.8 | | | | |
| Water (solvent)* | 7.1 | 6.5 | 8.3 | 8.6 | 8.8 | 7.1 | 7.1 | 6.8 | 8.3 |
| Hydrophobic | 18.6 | 18.7 | 18.3 | 18.3 | 18.2 | | | 46.6 | |
| Material A | | | | | | | | | |
| Hydrophobic | | | | | | 18.6 | | | |
| Material B | | | | | | | | | |
| Hydrophobic | | | | | | | 18.6 | | |
| Material C | | | | | | | | | |
| Hydrophobic | | | | | | | | | 20 |
| Material D | | | | | | | | | |
| 30-day | 54 | 33 | 6 | 38 | | | | | |
| hydrophobic | | | | | | | | | |
| material release | | | | | | | | | |
| (%) | | | | | | | | | |
| G′ (kPa) | 220 | 236 | 260 | 79.7 | | 428 | 595 | 194 | |
| G″ (Pa) | 1500 | 2700 | 27000 | 3000 | | 1900 | 6700 | 900 | |
| % Haze | 36 | 71 | 79 | 22 | | 30 | 25 | | |
| % Bio- | 85 | | | | | | | | |
| disintegration | | | | | | | | | |

*The water is removed during the synthesis process

Examples 1-3 show the formation of solid articles with three different epoxidized oils and using citric acid as cross-linker. Soybean epoxidized oil had an oxirane oxygen content (OOC) of 6.6%, castor epoxidized oil had an OOC of 6.8%, and linseed epoxidized oil had an OOC of 8%. A greater oxirane oxygen content indicates a greater number of epoxide moieties, and accordingly, a greater number of potential crosslinking sites. The results show that the higher the OOC % of the epoxidized oil, the higher the degree of cross-linking, and therefore the slower the release of hydrophobic material.

Examples 1, 4 and 5, show the formation of solid articles comprising epoxidized soybean oil, using three different acids as crosslinkers. The smaller the size of the crosslinker molecule, the tighter the crosslinking and the slower the release of hydrophobic material. The use of a tricarboxylic acid is more weight efficient for the same amount of cross-linking. These results confirm that the release rate of any particular hydrophobic material from a solid article according to the invention may advantageously be tuned by adjusting the nature of the materials used to form the gel matrix of the solid article. Materials that result in increased or tighter crosslinking (e.g. smaller crosslinking molecules or molecules having a higher density of potential crosslinking sites) may be used if it is desired to reduce the release rate of a hydrophobic material, while materials that result in reduced, or less tight, crosslinking may be used if it is desired to increase the release rate of a hydrophobic material.

Examples 6-9 show the versatility of hydrophobic materials that the solid article can be prepared with, and confirm that it is possible to prepare solid articles according to the invention with a range of hydrophobic materials having different properties.

All of the solid articles prepared above have solid gel properties, as confirmed by the G′ and G″ values.

Epoxidized soybean oil typically results in solid articles having a lower haze %, which may be perceived as providing aesthetic benefits.

### Examples 10-12

Table 2 lists solid articles according to the invention, which incorporate essential oils as the hydrophobic material and were prepared according to General Synthetic Method 1.

TABLE 2

| | Example No. | | |
|---|---|---|---|
| Raw Materials (wt. %) | 10 | 11 | 12 |
| Epoxidized soybean oil | 67.2 | 67.2 | 67.2 |
| Citric acid | 7.1 | 7.1 | 7.1 |
| Water (solvent)* | 7.1 | 7.1 | 7.1 |
| Cinnamon Oil | 18.6 | | |
| Peppermint Oil | | 18.6 | |
| Spearmint Oil | | | 18.6 |
| 30-day hydrophobic material release (%) | 33% | 49% | 56% |

*The water is removed during the synthesis process

### Examples 13-16

Examples 13-16 were prepared according to General Synthetic Method 1 with the materials set out in Table 3 below. However, the Examples 13-16 were unable to form a gel.

TABLE 3

| | Example No. | | | |
|---|---|---|---|---|
| Raw Materials (wt. %) | 13 | 14 | 15 | 16 |
| Epoxidized soybean oil | 54.1 | 39.7 | 66.2 | 71.5 |
| Succinic Acid | 5.3 | 15.5 | | |
| Trimesic Acid | | | 7.7 | |
| Polyacrylic Acid | | | | 8.5 |
| Solvent | 25.8 | 31 | 7.7 | |
| | (water) | (1:1 mix of | (water) | |
| | | water:acetone) | | |
| Hydrophobic Material A | 14.8 | 13.8 | 18.5 | 20 |

Without being bound by theory, it is believed that Examples 13 to 15 did not produce a gel due to poor solubility of the respective acids in water. Nevertheless, it is expected that solid articles including succinic acid and trimesic acid may be produced using an alternative solvent, in which the acids have a higher solubility, such as DMSO.

Without being bound by theory, it is believed that Example 16 did not produce a gel due to insufficient crosslinking of the epoxidized soybean oil by polyacrylic acid.

### Comparative Example 17

A solid article based on a polyurethane type polymer gel (Comparative Example 17) was prepared according to methods disclosed in US11173224B2 using castor oil and Desmodur® as a crosslinking agent, and having 20 wt. % of Hydrophobic Material A on a dry weight basis.

The 30-day hydrophobic material release for Comparative Example 17 was 57%. The bio-disintegration of Comparative Example 17 was 14%.

### Comparison Between Examples and Comparative Example

The 30-day hydrophobic material release for Comparative Example 17 was comparable to the hydrophobic material release obtained in Examples 1, 11 and 12. This confirms that the solid articles according to the invention may provide comparable release of hydrophobic material to prior art solid articles.

The bio-disintegration of Example 1 (based on a polyester matrix prepared from an epoxidized oil and a multivalent carboxylic acid) was advantageously much higher than that for Comparative Example 17 (based on a polyurethane polymer matrix). This confirms the excellent and improved bio-disintegration of the solid articles according to the invention.

It is expected that all of the Examples of the invention will have improved bio-disintegration as compared to solid articles prepared from a polyurethane matrix (e.g. Comparative Example 17), because the nature of the crosslinks in the polymer gel matrix for each Example is based on the same polyester chemistry as Example 1. Therefore, it is expected that all solid articles according to the current invention will have excellent bio-disintegration.

As explained above, the results also show that the release rate of hydrophobic material may be tuned by changing the materials from which the solid article is formed. This can be used to increase or decrease the release rate, as may be desired for a certain application. While the 30-day hydrophobic material release rates for Hydrophobic Material A varied significantly for Examples 1-4 (less variation was observed for Examples 10-12), this is because the nature of the crosslinking and polymer microstructure in each of these Examples is different. A skilled person will appreciate that other hydrophobic materials may be used in the solid articles of Examples 1 to 4 and provide higher or lower release rates as may be desired for a particular application. Therefore, the low release rate for Example 3 is not a disadvantage of the invention, and highlights the ability of the invention to provide a wide range of release rates, which may be desired for highly volatile materials.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A solid article for sustained release of one or more hydrophobic materials, comprising at least 5 wt. % of one or more hydrophobic materials embedded in a gel matrix,
wherein said gel matrix is formed from a chemically cross-linked polyester material.

2. The solid article of claim 1, wherein said chemically cross-linked polymeric material is a polyester obtainable by cross-linking an epoxidized vegetable oil with a polyfunctional carboxylic acid.

3. The solid article of claim 2, wherein said epoxidized vegetable oil comprises molecules having more than one (e.g. from 2 to 8, such as from 2 to 6) epoxy groups, and where the epoxidized vegetable oil is characterized by an oxirane oxygen content of at least 5% (e.g. at least 6%);
wherein optionally said epoxidized vegetable oil is selected from the group consisting of epoxidized linseed oil, epoxidized soybean oil, epoxidized castor oil, epoxidized rapeseed oil, epoxidized vernonia oil, vernonia oil, epoxidized corn oil, epoxidized cottonseed oil, epoxidized canola oil, epoxidized grape seed oil, epoxidized poppy seed oil, epoxidized tung oil, epoxidized sunflower oil, epoxidized safflower oil, epoxidized walnut oil, and any combinations thereof;
and wherein more optionally said epoxidized vegetable oil is epoxidized soybean oil.

4. The solid article of claim 2, wherein said polyfunctional carboxylic acid is soluble in at least one non-alcoholic (e.g. non-nucleophilic) solvent;
optionally wherein said polyfunctional carboxylic acid is selected from the group consisting of citric acid, maleic acid, malonic acid, itaconic acid, fumaric acid, succinic acid, methyl succinic acid, malic acid, phthalic acid, tartaric acid, citraconic acid, furan dicarboxylic acid, and any combinations thereof;
more optionally wherein said polyfunctional carboxylic acid is selected from the group consisting of citric acid, maleic acid, itaconic acid, fumaric acid, succinic acid, methyl succinic acid, malic acid, tartaric acid, citraconic acid, furan dicarboxylic acid, and any combinations thereof; and
more optionally still wherein said polyfunctional carboxylic acid is citric acid.

5. The solid article according to claim 2, wherein said polyester is obtainable from a crosslinking reaction carried out in the presence of a non-alcoholic solvent;
wherein optionally said non-alcoholic solvent is selected from the group consisting of water, acetone, toluene, and any combinations thereof; and
wherein optionally said non-alcoholic containing solvent is water.

6. The solid article according to claim 1, wherein the solid article is self-supporting.

7. The solid article according to claim 1, wherein the solid article comprises from 5 to 85 wt. % of the one or more hydrophobic materials, based on the total mass of the solid article,
optionally from 10 to 75 wt. %,
more optionally from 15 to 60 wt. %,
further optionally from 20 to 55 wt. %,
more optionally still from 20 to 50 wt. %.

8. The solid article according to claim 1, wherein the one or more hydrophobic materials have a logP of from 0.01 to 6.5 on a weighted average basis,
optionally from 0.5 to 5.5,
more optionally from 1.5 to 5.5,
further optionally from 2 to 5.

9. The solid article according to claim 1, wherein the one or more hydrophobic materials comprise one or more volatile hydrophobic materials,
optionally wherein the one or more hydrophobic materials comprise from 20 to 100 wt. % of one or more volatile hydrophobic materials,
optionally wherein the one or more hydrophobic materials comprise from 40 to 100 wt. % of one or more volatile hydrophobic materials,
optionally wherein the one or more hydrophobic materials comprise from 60 to 100 wt. % of one or more volatile hydrophobic materials,
optionally wherein the one or more hydrophobic materials comprise from 80 to 100 wt. % of one or more volatile hydrophobic materials.

10. The solid article according to claim 9, wherein the one or more volatile hydrophobic materials each have a boiling point of less than or equal to 450° C. at atmospheric pressure,
more optionally wherein the volatile hydrophobic material has boiling point of from 60° C. to 400° C. at atmospheric pressure,
further optionally wherein the volatile hydrophobic material has boiling point of from 75° C. to 380° C. at atmospheric pressure.

11. The solid article according to claim 9, wherein the one or more volatile hydrophobic materials each have a vapor pressure of at least 10⁻⁶ Torr at 25° C. and atmospheric pressure,
optionally wherein the volatile hydrophobic material has a vapor pressure of at least 10⁻⁵ Torr at 25° C.,
more optionally wherein the volatile hydrophobic material has a vapor pressure of at least 10⁻⁴ Torr at 25° C.

12. The solid article according to claim 1, wherein the one or more hydrophobic materials comprise a material selected from the group consisting of a perfume, an insect repellent, an essential oil, a functional perfume component (FPC), an aesthetic, a bioactive, a malodor counteractant, and mixtures thereof.

13. The solid article according to claim 1, wherein the solid article is configured to release at least 50 wt. % of the hydrophobic material when stored at 25° C. for 30 days at atmospheric pressure,
optionally wherein the solid article is configured to release at least 70 wt. % of the hydrophobic material when stored at 25° C. for 30 days at atmospheric pressure.

14. A method of making a solid article according to claim 1, comprising the steps:
(i) mixing an epoxidized vegetable oil with a polyfunctional carboxylic acid in a solvent;
(ii) adding the one or more hydrophobic materials and mixing until homogeneous; and
(iii) allowing the mixture to rest for at least 4 hours.

15. The method of claim 14, wherein step (i) is performed at a temperature of from 60 to 150° C.,
optionally from 80 to 120° C.

16. The method of claim 14, wherein step (i) is performed until the mixture has a viscosity of from 30 to 80 cP.

17. The method of any one of claims 14, wherein step (ii) is performed at a temperature of from 0 to 50° C.,
optionally from 20 to 40° C.

18. The method of any one of claims 14, wherein step (iii) is performed at a temperature of from 35 to 75° C. for a period of from 4 to 48 hours,
optionally 12 to 36 hours,
more optionally 18 to 30 hours.

19. Use of a solid article according to claim 1 for:
(a) the sustained release of a hydrophobic material selected from the group consisting of a perfume, an insect repellant, and combinations thereof; or
(b) the sustained release of a hydrophobic material in a damp environment, optionally wherein the damp environment is selected from the group consisting of a washing machine, a dishwasher, a shower and a sink.
